(19) United States
(12) Patent Application Publication
(10) Pub. No.: US 2025/0108330 A1
(43) Pub. Date: Apr. 3, 2025

(54) ELECTROCHEMICAL SYSTEM FOR CARBON DIOXIDE CAPTURE

(71) Applicant: 12272580 Canada Inc., Cow Bay (CA)

(72) Inventor: Qinhong Cai, Ottawa (CA)

(21) Appl. No.: 18/904,990

(22) Filed: Oct. 2, 2024

Related U.S. Application Data
(63) Continuation-in-part of application No. PCT/CA2024/050385, filed on Mar. 27, 2024.
(60) Provisional application No. 63/455,646, filed on Mar. 30, 2023.

(51) Int. Cl. B01D 53/96; B01D 53/62; B01D 53/78; C12P 5/02; C25B 1/16; C25B 5/00; C25B 9/23; C25B 11/052; C25B 11/065; C25B 11/079; C25B 13/08; H01M 8/16

(52) U.S. Cl. CPC B01D 53/965; B01D 53/62; B01D 53/78; C12P 5/023; C25B 1/16; C25B 5/00; C25B 9/23; C25B 11/052; C25B 11/065; C25B 11/079; C25B 13/08; H01M 8/16; B01D 2251/304; B01D 2251/604; B01D 2257/104; B01D 2257/504; B01D 2258/0283; B01D 2258/06

(57) ABSTRACT

The present application relates to an electrochemical system and method for capturing carbon dioxide, regenerating purified carbon dioxide and/or converting the captured carbon dioxide to biomethane, syngas, formic acid, and combinations thereof. More specifically, the present application relates to an electrochemical cell having an air cathode proton or cation exchange membrane and an abiotic or biotic anode wherein captured carbon dioxide is reduced to carbonates that are regenerated as purified carbon dioxide or transferred to another chemical production module. The regenerated carbon dioxide can be purified, compressed, or stored for later use. The present application includes volatile fatty acids sourced from carbohydrate rich waste. The present application also includes the ability to regenerate caustic absorbents used in initial carbon dioxide capture.

### CROSS-REFERENCE TO RELATED APPLICATION

The present application is a continuation-in-part of PCT/CA2024/050385 filed Mar. 27, 2024, which claims the benefit of priority of U.S. Provisional Patent Application No. 63/455,646, which was filed Mar. 30, 2023, the contents of which are incorporated herein by reference in their entirety.

### FIELD

The present application is in the field of electrochemistry and carbon dioxide capture. More specifically, the present application relates to an electrochemical system and method for capturing carbon dioxide, regenerating purified carbon dioxide and/or converting the captured carbon dioxide to methane.

### BACKGROUND

The concentration of atmospheric carbon dioxide is rapidly increasing. From the combustion of fossil fuels, coal, oil and natural gas, increased carbon dioxide levels are threatening life across the globe. Carbon capture and regeneration is a promising strategy to reduce greenhouse effects by reducing carbon dioxide emissions. Electrochemical conversion of carbon dioxide into fuels or other value-added chemicals from exhaust streams are vital to reducing greenhouse gas emissions.

Carbon dioxide absorbents and desorption technologies are pivotal to achieving this aim. Common carbon dioxide absorbents include amines such as monoethanolamine (MEA) and alkoxide salts of sodium, lithium, and potassium. NaOH as a CO₂ absorbent outperforms organic absorbents, such as MEA with a faster reaction kinetics (Gong et al., 2006; Ye et al., 2015) and a higher absorption efficiency (Peng et al., 2012). It offers higher stability, lower cost, and lower environmental impact than amine solutions (Penders-van Elk et al., 2013; Ye et al., 2015). However, the application of NaOH is facing the challenge of CO₂ desorption from the capture solution. Existing commercial routes utilize Ca(OH)₂ to form CaCO₃ from Na₂CO₃, which are then incinerated at 674° C. to release CO₂ (Keith et al., 2018). Other than using the complex calcium-based thermochemical cycle, bipolar membrane electrodialysis (BPMED) is an alternative with the advantage of direct use of renewable energy, avoiding high temperature conditions, phase transitions and solid handling (Sabatino et al., 2020). Valluri and Kawatra (2021) found BPMED required less energy than that required for thermal regeneration. However, the high cost of the bipolar membrane (BPM) is a major constraint.

As such, there is a need to provide improved electrochemical systems for carbon dioxide capture that would overcome at least some of the drawbacks of existing technologies, that would be cost-effective, energy-efficient and versatile.

### SUMMARY

It has been shown herein that systems of the present application provide for cost effective and efficient alternatives to the expensive BPM-based regeneration of caustic solution for CO₂ capture. The processes of the present application further provide for energy efficient and versatile methods.

Accordingly, the present application includes an electrochemical system for capturing carbon dioxide from a gas stream, the system comprising: an electrochemical cell comprising an anode chamber and a cathode chamber, where the cell contains an electrolyte solution; a cathode exposed to the electrolyte solution in the cathode chamber; an anode exposed to the electrolyte solution in the anode chamber; wherein the electrochemical cell is connectable to a power supply for electrically connecting the anode and the cathode to apply a potential difference between the anode and the cathode; a proton or cation exchange membrane separating the anode chamber and the cathode chamber allowing alkali metal ions formed at the anode to diffuse through the proton or cation exchange membrane towards the cathode to react with hydroxide ions to form an alkali metal base, a collection chamber to collect the alkali metal base from the cathode chamber; a source of the gas stream comprising carbon dioxide for contacting with the alkali metal base such that the carbon dioxide reacts with the alkali metal base to form a carbonate, and thus capture the carbon dioxide from the gas stream.

The present application also includes a method for capturing carbon dioxide from a gas stream is provided, comprising: applying a potential difference between an anode and a cathode in an electrochemical cell containing an electrolytic solution such that alkali metal ions are formed in the electrolytic solution at the anode, permitting the alkali metal ions to diffuse through a proton or cation exchange membrane between the anode and the cathode, towards the cathode to react with hydroxide ions to form an alkali metal base, separating the alkali metal base from the electrolytic solution, contacting the gas stream comprising carbon dioxide with the alkali metal base such that the carbon dioxide reacts with the alkali metal base to form a carbonate, thus capturing the carbon dioxide from the gas stream.

Accordingly, the present application further includes an electrochemical system for capturing carbon dioxide from a gas stream and generating methane, the system comprising: an electrochemical cell comprising an anode chamber and a cathode chamber, where the cell contains an electrolyte solution; a cathode exposed to the electrolyte solution in the cathode chamber; an anode exposed to the electrolyte solution in the anode chamber; a power supply electrically connected to the anode and the cathode configured to apply a potential difference between the anode and the cathode; a proton or cation exchange membrane separating the anode chamber and the cathode chamber allowing alkali metal ions formed at the anode to diffuse through the proton or cation exchange membrane towards the cathode to react with hydroxide ions to form an alkali metal base, a collection chamber to collect the alkali metal base from the cathode chamber; a source of the gas stream comprising carbon dioxide for contacting with the alkali metal base such that the carbon dioxide reacts with the alkali metal base to form a carbonate, and thus capture the carbon dioxide from the gas stream, a deoxygenation unit to remove dissolved oxygen from the carbonate solution to produce a deoxygenated carbonate solution, and a biomethane production module configured to receive the deoxygenated carbonate solution, the biomethane production module comprising biocatalysts to react with the carbonate to produce methane.

The present application also includes a method for generating methane by capturing carbon dioxide from a gas stream is provided, the method comprising: applying a potential difference between an anode and a cathode in an electrochemical cell containing an electrolytic solution such that alkali metal ions are formed in the electrolytic solution at the anode, permitting the alkali metal ions to diffuse through a proton or cation exchange membrane between the anode and the cathode, towards the cathode to react with hydroxide ions to form an alkali metal base, separating the alkali metal base from the electrolytic solution, contacting the gas stream comprising carbon dioxide with the alkali metal base such that the carbon dioxide reacts with the alkali metal base to form a carbonate, thus capturing the carbon dioxide from the gas stream; deoxygenating the carbonate with an aerobic bacterium, transferring the carbonate to biocatalysts for generating methane.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF DRAWINGS

The embodiments of the application will now be described in greater detail with reference to the attached drawings in which:

FIG. 1 shows a caustic recovery ACPEM or ACCEM system layout, according to exemplary embodiments of the application.

FIG. 2 shows results for a ACPEM or ACCEM system with an abiotic anode, according to exemplary embodiments of the application where A) illustrates the electrochemical principle; B) is a graph of [Cations] at various pH levels; C) is a graph of reaction potentials at cathode and anode at different pH values, with arrows showing cell voltage requirements; and D) is a graph of [HCO₃—] in solution at various pH levels (60% CO₂ headspace and 25° C.).

FIG. 3. is a schematic diagram of reactions in a ACPEM or ACCEM system with abiotic anode, according to exemplary embodiments of the application.

FIG. 4. is a schematic diagram of a ACPEM or ACCEM system with an abiotic anode, according to exemplary embodiments of the application.

FIG. 5. illustrates the electrochemical principle of a ACPEM or ACCEM system with a biotic anode, according to exemplary embodiments of the application.

FIG. 6. is a schematic diagram of reactions in a ACPEM or ACCEM system with biotic anode, according to exemplary embodiments of the application.

FIG. 7. is a schematic diagram of a ACPEM or ACCEM system with a biotic anode, according to exemplary embodiments of the application.

FIG. 8. is a schematic diagram illustrating energy consumption of CO₂ capture coupled with bioelectrochemical methane production (BEMP) according to exemplary embodiments of the application, compared with traditional carbon capture and subsequent transformation processes, according to exemplary embodiments of the application.

FIG. 9. is a schematic diagram of a ACPEM or ACCEM system with a biotic anode coupled to a BEMP module, according to exemplary embodiments of the application.

FIG. 10. is a schematic of reactions in a Na₂CO₃/NaHCO₃ based BEMP coupled with carbon capture system, according to exemplary embodiments of the application.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

### II. Systems of the Application

The systems of the application have been shown to serve as a cost effective and efficient alternative to the expensive BPM-based regeneration of caustic solution for CO₂ capture.

Accordingly, the present application includes an electrochemical system for capturing carbon dioxide from a gas stream, the system comprising: an electrochemical cell comprising an anode chamber and a cathode chamber, where the cell contains an electrolyte solution; an air cathode exposed to the electrolyte solution in the cathode chamber; an anode exposed to the electrolyte solution in the anode chamber; a power supply electrically connected to the anode and the cathode configured to apply a potential difference between the anode and the cathode; a proton or cation exchange membrane separating the anode chamber and the cathode chamber allowing alkali metal ions formed at the anode to diffuse through the proton or cation exchange membrane towards the cathode to react with hydroxide ions to form an alkali metal base, a collecting chamber to collect the alkali metal base from the cathode chamber; a source of the gas stream comprising carbon dioxide for contacting with the alkali metal base such that the carbon dioxide reacts with the alkali metal base to form a carbonate, and thus capture the carbon dioxide from the gas stream.

In some embodiments, the system of the application comprises an electrochemical system with a three-phased air cathode and proton exchange membrane assembly (ACPEM) or a three-phased air cathode and cation exchange membrane assembly (ACCEM) for cost-effective caustic regeneration, in which H⁺ are continuously generated through anodic reaction and accumulated in the anode chamber, whereas the alkali metal cations cross the PEM or CEM to form the alkali metal base with OH-resulting from proton consuming cathodic reactions. The system generates pH differences between electrodes via hindering proton or cation transfer through PEM or CEM leveraging the abundant alkali metal cations in the CO₂ capture solution. This design can incorporate either an abiotic anode (oxygen evolution reaction) or a biotic anode (acetate oxidation reaction).

In some embodiments, the cathode has a flow inlet and a flow outlet. In some embodiments, the cathode flow inlet is configured to receive the electrolyte solution and the cathode flow outlet is configured to transfer the alkali metal base generated away from the cathode towards the collecting chamber.

In some embodiments, the anode has a flow inlet and flow outlet. The anode flow inlet may be configured to receive the electrolyte solution or the captured CO₂ solution that subsequently reacts with H⁺ to release the carbon dioxide. The anode flow outlet may be configured to allow release of the processed electrolyte solution or captured CO₂ solution. Built up carbon dioxide in the anode headspace is released to a carbon reservoir or to a further processing unit.

In some embodiments, the anode is an abiotic anode wherein an oxygen evolution reaction takes place. In some embodiments, the anode is a biotic anode wherein an acetate oxidation reaction is catalyzed by respiring microorganisms takes place within the anode chamber. In some embodiments, the microorganisms may include Geobacter spp. or Shewanella spp. In some embodiments, the acetate ions are sourced from volatile fatty acids in a separate acetate reactor before being transferred to the anode. In some embodiments, the fatty acids in the acetate reactor react with carbonate to release carbon dioxide and form salts comprising sodium acetate. In some embodiments, the salts are directed to a ACPEM, ACCEM or BEMP. In some embodiments, the acetate ions are sourced from carbohydrate rich waste. In some embodiments, the acetate ions are sourced from a combination of fermentation and anaerobic digestion processes in a cost-effective manner. In some embodiments, the acetate ions are sourced from forestry and crop waste, wherein the combination of fermentation and anaerobic digestion processes generate acids while allowing residual material to be easily dried and compressed as pellets. In some embodiments, the acetate ions are sourced from wastewater from Fischer-Tropsch reaction, biomass pyrolysis, sugar processing, potato processing, etc. In some embodiments, the acetate ions are sourced from ethanol production to help reduce the carbon intensities of fuel ethanol. In some embodiments, the acetate ions are sourced from potato processing, fruit processing, or paper mill industry waste. In some embodiments, the acetate ions formed in the acetate reactor are subjected to deoxygenation before being transferred to the anode. In some embodiments, the acetate ions formed in the acetate reactor are transferred to a deoxygenation system before being transferred to the anode.

In some embodiments, the solution in the anode chamber has a pH from about 5 to about 7, or about 6 to about 7, or about 7.

In some embodiments, the solution in the cathode chamber has a pH of about 12 to about 14, or about 13 to about 14, or about 14.

In some embodiments, the cathode is an air cathode. In some embodiments, the air cathode is based on a carbon support such as carbon felt, activated carbon, black carbon, carbon cloth, or VULCAN carbon support having high porosity to facilitate air, water, and electrode interactions. In some embodiments, the carbon support is a nitrogen doped carbon support with a catalyst comprising Pt, Fe or Co. In some embodiments, the air cathode comprises an embedded current collector that increases power generation of fuel cells by decreasing cathodic charge transfer impedance. In some embodiments, the embedded current collector is made of an acid and alkaline resistant material comprising titanium or stainless steel.

In some embodiments, the cathode comprises a film including MnO₂ deposited onto carbon felt. In some embodiments, the film is deposited via electrodeposition techniques.

In some embodiments, the cathode is a hydrogen evolution cathode. In some embodiments, the hydrogen evolution reaction is catalyzed by Pt.

In some embodiments, the electrolyte solution is an aqueous solution of the formula XₙY wherein X is Na⁺, K⁺, or Li⁺, Y is SO₄²⁻, CO₃²⁻, HCO₃⁻, and n is 1 or 2 or a combination thereof.

In some embodiments, the power supply is electrically connected to the anode and the cathode configured to apply a potential difference between the anode and the cathode. In some embodiments, the power supply is an external electric source or is recycled from a component of the system. In some embodiments, the power supply is recycled when the anode of the ACPEM or ACCEM with biotic anode is configured to be connected to the cathode of a BPMED; and the cathode of the ACPEM or ACCEM with biotic anode is configured to be connected to the anode of a BPMED. In some embodiments, the potential difference is from about −0.9V to about 0.75V, or about −0.85V to about 0.7V, or about −0.8V to about 0.65V, or about −0.75 to about 0.6V, or about −0.7V to about 0.55V.

In some embodiments, the proton or cation exchange membrane is selected from pure polymer membranes and composite membranes. The proton or cation exchange membrane may be a semipermeable membrane comprising perfluorinated sulfonic acid ionomer structures. The PEM/CEM may be selected from Nafion®, Flemion®, Aciplex®, Pemion®, Cemion®, Fumapern®, Fujifilm® as well as sulfonated poly (ether ether) (SPEEK) and PVA-Nafion-borosilicate.

In some embodiments, the alkali metal ion is Na⁺, K⁺, Li⁺ or combinations thereof.

In some embodiments, the gas stream is sourced from the combustion of fossil fuels, coal, oil and/or natural gas, for example from an energy production process.

In some embodiments, the gas stream is air, exhaust gas, flue gas produced during the combustion of fossil fuels, coal, oil and/or natural gas for energy production.

In some embodiments, the collecting chamber is an external container used to collect the generated alkali metal base solution. In some embodiments, the collecting chamber is configured to allow the liquid to be directed in or out of the ACPEM, ACCEM or BEMP using a transferrer. In some embodiments, the transferrer operates by mechanical action or gravity. In some embodiments, the alkali metal base in the collecting chamber is transferred to a gas contactor housed in a carbon capture unit to contact the gas stream comprising carbon dioxide such that the carbon dioxide reacts with the alkali metal base to form a carbonate, and thus capture the carbon dioxide from the gas stream. In some embodiments, the carbonates formed therein are stored in a carbon reservoir for further processing. In some embodiments, further processing comprises CO₂ release, deoxygenation, combinations thereof or any systems requiring a CO₂ input. In some embodiments, the carbon capture unit is a system configured to allow efficient gas-liquid contact, i.e. contact between the gas stream and the alkali metal base solution.

In some embodiments, CO₂ release is carried out by returning the carbonates solution to the anode chamber to react with the H⁺ generated at the anode, and thus release the CO₂. In some embodiments, the released CO₂ is directed to a gas processing system comprising a carbon dioxide storage container for optionally dewatering, purification and/or purity analysis before being compressed for transportation, utilization, or storage.

In some embodiments, the system further comprises a transferrer for bringing the alkali metal base from the collecting chamber to a gas contactor of a carbon capture unit configured to bring the gas stream in contact with the alkali metal base. In some embodiments, the transferrer may also bring captured carbon dioxide in the form of carbonate that is generated in a ACPEM or ACCEM biotic anode system to a deoxygenation system and/or subsequently to the anode to react the carbonate with hydrogen ions and release carbon dioxide. In some embodiments, the transferrer may bring the captured carbon dioxide to a further processing system selected from purification and compression processes. In some embodiments, the transferrer operates by mechanical action or gravity. In some embodiments, the transferrer is selected from a peristatic pump, a pressure driven flow control pump, a diaphragm pump, a centrifugal pump or combinations thereof. In some embodiments, the transferrer is operatively connected at each stage of the system for transferring the respective solutions/products to a next stage. In some embodiments, fluid communication between each stage of the system is provided by suitable tubes or pipes, or the like.

Without being bound to theory, the versatility of the system of the application allows it to work in conjunction with other systems requiring a CO₂ input, such as a bioelectrochemical methane production (BEMP) module, wherein the captured CO₂ solution in the form of carbonates can be introduced to BEMP after pH monitoring and deoxygenation. In some embodiments, the pH is monitored with a pH meter or the like, to determine the reaction end point. The introduction of CO₂ in bicarbonate form to BEMP may present at least one of four (4) advantages: 1) realization of direct synergy between carbon capture and BEMP.

In some embodiments, before the carbonate solution generated from CO₂ capture can be applied to a biotic anode or a BEMP, the dissolved oxygen level may need to be reduced to protect oxygen sensitive anode respiring bacteria and methanogens. In some embodiments, a biological deoxygenation unit is designed and incorporated in the system of the application. In some embodiments, the deoxygenation unit takes advantage of Halomonas alkaliphila, an aerobic bacterium with high XHCO₃/X₂CO₃ tolerance (where X is alkali metal cation), to consume dissolved oxygen in the capture solution.

In some embodiments, the deoxygenation unit comprises carbonate tolerant aerobic bacterium including Halomonas alkaliphila or other carbonate tolerant bacteria.

Overall, the ACPEM/ACCEM of the present application with an abiotic or biotic anode may serve as an alternative to the expensive BPM-based regeneration of caustic solution for CO₂ capture, in which low-cost PEM/CEM and MnO₂ coated carbon felt replace expensive BPM and associated catalysts. Without being bound to theory, the air cathode instead of hydrogen evolution cathode allow lower onset voltage and energy consumption. When using a biotic anode, volatile fatty acids (VFA) generated from carbohydrate rich waste can be incorporated to regenerate the caustic solution, release captured and generated CO₂, and generate current/voltage to support other energy consuming processes. Coupling CO₂ capture coupled with BEMP via direct reduction of bicarbonate provides a synergic solution to both technologies. The combined process of ACPEM/ACCEM of the present application with biotic anode and BEMP was achieved, coupling CO₂ capture and biomethane production with superior pH management, biocatalyst efficiency, and product mixture (CO₂ and CH₄) separation to traditional anaerobic digestion. Lastly, a cost-effective biological deoxygenation unit may be incorporated to protect oxygen sensitive methanogens and anode respiring microbes in the downstream processes.

### III. Methods and Uses of the Application

The present application further provides methods and uses of an electrochemical system to capture carbon dioxide, regenerate a purified stream of carbon dioxide and/or couple captured carbon dioxide as carbonates to a module to produce purified methane, syngas, formic acid, and combinations thereof.

Accordingly, the present application includes a method for capturing carbon dioxide from a gas stream, the method comprising: generating an alkali metal base in an electrochemical cell comprising an anode chamber and a cathode chamber, where the cell contains an electrolyte solution; an air cathode exposed to the electrolyte solution in the cathode chamber; an anode exposed to the electrolyte solution in the anode chamber, and a proton/cation exchange membrane separating the anode chamber and the cathode chamber, by applying a potential difference between the anode and the cathode such that alkali metal ions are formed at the anode and diffuse through the proton/cation exchange membrane towards the cathode to react with hydroxide ions to form the alkali metal base, transferring the alkali metal base from the cathode chamber to a collecting chamber; contacting the gas stream comprising carbon dioxide with the alkali metal base such that the carbon dioxide reacts with the alkali metal base to form a carbonate, thus capturing the carbon dioxide from the gas stream.

In some embodiments, the transferring of the alkali metal base from the cathode chamber to the collecting chamber is carried out by a transferrer.

In some embodiments, the method further comprises storing the generated carbonate solution in a carbon reservoir container. In some embodiments, the method further comprises transferring the carbonate solution to the anode chamber to be reacted with H⁺ and release the carbon dioxide. In some embodiments, the method further comprises transferring the carbonate solution to an external container housing volatile fatty acids wherein the acids react with carbonate to release carbon dioxide. In some embodiments, the method further comprises transferring the carbonate solution to a deoxygenation unit. In some embodiments, the method further comprises transferring the carbonate solution or deoxygenated carbonate solution to a further processing unit. In some embodiments, the further processing unit comprises purifying or compressing the carbonate solution.

### EXAMPLES

The following non-limiting examples are illustrative of the present application.

### Example 1—ACPEM or ACCEM Assembly

In principle, ACPEM/ACCEM utilizes the abundant Na⁺ (e.g. 4 M in abiotic anode) in the anode chamber to compete with scarce protons (H⁺) (e.g. 10⁻⁷ M at neutral pH) for the PEM/CEM. The competition hinders the crossover of H⁺ through the PEM. H⁺ continuously generated through anodic reaction is then accumulated in the anode chamber, whereas Na⁺ cross the PEM/CEM to form NaOH with OH⁻ resulting from proton/cation consuming cathodic reactions. The end-result is higher pH at the cathode and lower pH at the anode. In this design, instead of using a hydrogen evolution reaction, which is typically used in BPMED at the cathode, an air cathode is utilized to provide a thermodynamically favorable conditions coupling with either an abiotic oxygen evolution reaction or a biotic acetate oxidation reaction in the anode chamber. When compared with the work of Bouwman et al. (2018), which generates NaOH along with their reduced products (e.g. methanol and formic acid) at the cathode, this design avoids the downstream separation of products and the regenerated caustic solution.

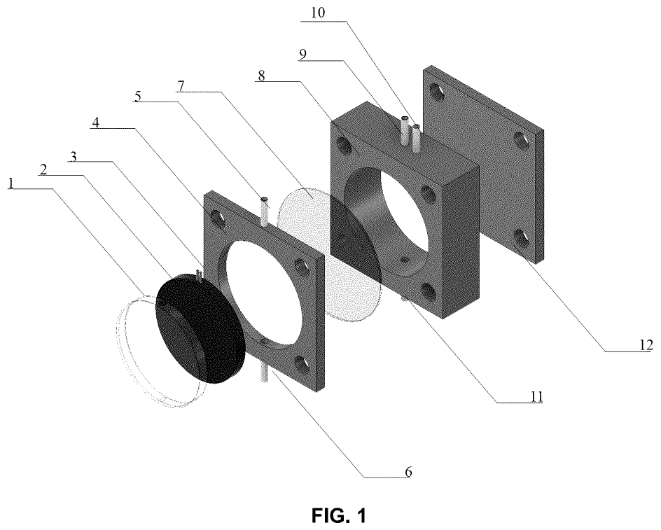

As illustrated in FIG. 1, a caustic recovery ACPEM/ACCEM assembly layout is depicted having a parafilm cover 1, an MnO₂-deposited carbon felt (cathode) 2, an embedded current collector (stainless steel) 3, a cathode chamber 4, a cathode flow inlet 5, a cathode flow outlet 6, a proton/cation exchange membrane 7, an anode chamber 8 (abiotic or biotic electrode not shown), an anode flow inlet 9, an anode gas outlet 10, an anode flow outlet 11, and an end plate 12.

Further illustrated in FIG. 1, a PEM/CEM (e.g. Nafion® 117) is directly pressed against the air cathode, while separating the anode chamber from the air cathode. Several strategies are available to create a low-cost Nafion® based PEM (Tiwari et al., 2016). The air cathode was based on carbon felt with high porosity to facilitate the three phases (air/water/electrode) interactions. Carbon felt was first treated with 10% H₂O₂ solution at 90° C. for 3 h followed by washing with 10% HCl solution for 1 h at 90° C. to remove impurities and improve the hydrophilicity/wettability of the carbon felt (Zhou et al., 2006). The carbon felt was then isolated and cleaned with distilled water before dried at room temperature. Subsequently, MnO₂ films were deposited onto the carbon felt via electrodeposition techniques using a two-electrode system, where carbon felt, Pt rod, and the 0.25 M Mn(CH₃COO)₂ solution were used as the working electrode, counter electrode, and electrolyte respectively. A controlled current of 30 mA was supplied for 60 min to complete the electrodeposition of MnO₂ onto the carbon felt (Zhang et al., 2015). The resulting MnO₂ coated carbon felt was found to effectively catalyze the oxygen reduction reaction with good ability to retain and distribute water within the catalytic matrix. A stainless-steel or Ti wire was used to serve as current collector within the cathode. The cathode was then embedded in the cathode chamber. A parafilm layer facing the air side was added to the cathode to avoid excessive water loss from evaporation, while allowing air to pass through for the oxygen reduction reaction. A small stream of water was applied to the inlet of the cathode chamber to carry the produced NaOH solution away from the cathode and towards the product collecting container. A short tubing and an intravenous fluid bag style container were used to limit the exposure of the generated NaOH to air, which would consume the alkalinity.

The ACPEM/ACCEM assembly can be integrated with either an abiotic anode catalyzing oxygen evolution reaction or a biotic anode catalyzing acetate oxidation. When compared with BPMED, which needs at least 0.83 V across a BPM at which water dissociation takes place when starting from a 1 M salt solution, ACPEM combined with an abiotic anode requires lower onset voltage from a thermodynamic point of view. When using a biotic anode, the reactions are spontaneous with electricity output.

### Example 1a—ACPEM or ACCEM with an Abiotic Anode

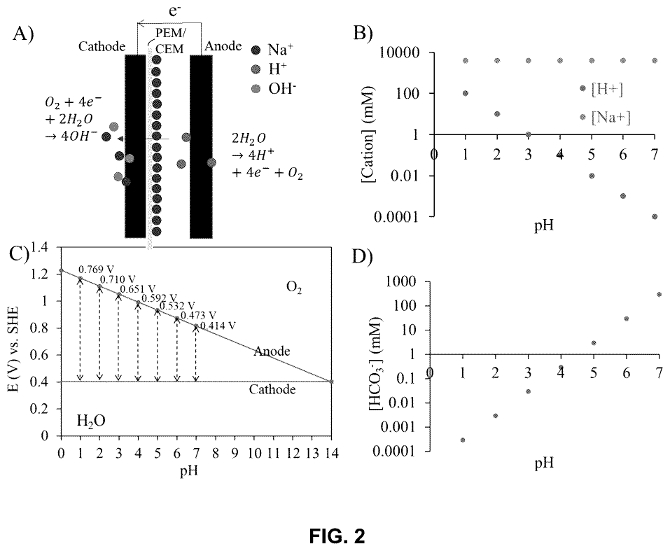

FIG. 2, A) shows the working principle of ACPEM/ACCEM and an abiotic anode.
At cathode: O₂+4e⁻+2H₂O→4OH⁻ at 1M NaOH⁻ E (V) vs SHE=0.401
At anode: 2H₂O→4H⁺+4e⁻+O₂ E (V) vs SHE will be based on the pH level in the anode chamber (FIG. 2, C)).

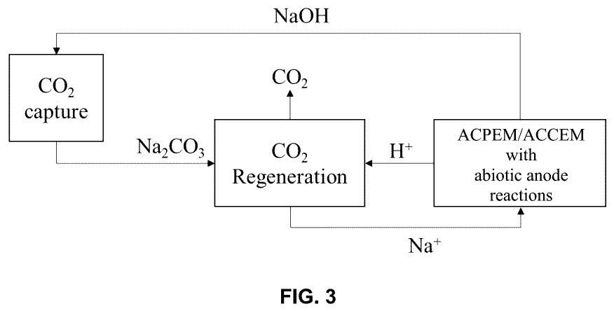

Without wishing to be bound to theory, PEM/CEM facilitated the transport of both Na⁺ and H⁺. When using 1 M Na₂SO₄ and 1 M Na₂CO₃ as the electrolyte, the concentration ratio of Na⁺/H⁺ was 4×10⁷. This large concentration difference limited the transportation of H⁺ through the PEM/CEM, which leads to acidification of the anode solution. In this design, as the anode can become an acidic environment, an oxygen evolution reaction catalyst that is acid tolerant was employed. An exemplary choice is a cost-effective and stable manganese oxybromide (Mn_{7.5}O₁₀Br₃) catalyst (Pan et al., 2022). The accumulated H⁺ will react with Na₂CO₃ generated from CO₂ capture to allow the release of CO₂ (FIG. 3). A deliberately selected pH range is used to optimize the efficiency of the system.

As shown in FIG. 2, B), without being bound to theory, the accumulation of H⁺ along with lower pH will narrow the gap between the concentration ratio of Na⁺/H⁺, which may render the mechanism less effective. Therefore, a relatively high pH value may be required in the anode chamber. Furthermore, as shown in FIG. 2 C) (based on the Nernst equation), the anode potential increases with decreasing pH, which leads to higher cell voltage inputs. From these two aspects, the anode pH would ideally be relatively higher. To directly use CO₃²⁻ in the anode to react with the H⁺ generated from oxygen evolution reaction will avoid the building up of free H⁺ and lower pH values. This allows efficient energy consumption (FIG. 2, C)) and selective transportation of Na⁺ (FIG. 2, B)). However, as the oxygen evolution reaction generates O₂ gas as a product, the released gas was in the form of a mixture of O₂ and CO₂. Forty to sixty percent of CO₂ in the mixture were observed in our experiments. The gas mixture stream can be processed through either 1) a well buffered high cell density bioprocess culturing a high oxygen uptake strain, which typically requires O₂ enriched air; or 2) a reversible oxygen absorption material such as the absorber prepared by Sundberg et al., (2014) to remove O₂ from the mixture and allow highly concentrated CO₂ to be collected.

In the CO₂ regeneration process, the constituents of the CO₂·H₂O/HCO₃⁻/CO₃²⁻ buffer system shifts with pH values. Higher pH values led to the presence of higher concentration of HCO₃⁻ and reduced the percentage of CO₂ released (FIG. 2, D)). The relationship between pH values and the constituents of the bicarbonate buffer system can be expressed through the Henderson-Hasselbalch equation (1):

$$pH = {pK}_{a {H}_{2} C {O}_{3}} + log ( \frac{[ {HCO}_{3}^{-} ]}{{k}_{H {CO}_{2}} \times {p}_{{CO}_{2}}} ) \qquad ( 1 )$$

where:
pK_{a H}_{₂}_{CO}_{₃} is the negative logarithm (base 10) of the acid dissociation constant of carbonic acid; [HCO₃⁻] is the concentration of bicarbonate in the solution; K_{H CO}_{₂} is the Henry's law constant reflecting the solubility of CO₂ in the solution. p_{CO}_{₂} is the partial pressure of CO₂ in the headspace.

During the CO₂ regeneration process, the headspace had much higher CO₂ concentration than air (˜60%), leading to a high p_{CO}_{₂}≈380 mmHg under atmospheric pressure. This made [HCO₃⁻] change significantly with changing pH values (FIG. 2, D)). For example, at pH=7, 176.24 mM of [HCO₃⁻] presented along with 14 mM of dissolved CO₂ in the form of H₂CO₃. A total of 190.24 mM of CO₂ equivalent was released from the solution. For 1 M Na₂CO₃ used for the CO₂ regeneration process, 38% of the CO₂ equivalent was not released under the atmospheric pressure. Therefore, without being bound to theory, a lower pH end point for CO₂ regeneration process is recommended, for example, around pH=5. At pH=5, only 3.15% of CO₂ equivalent remained in the solution.

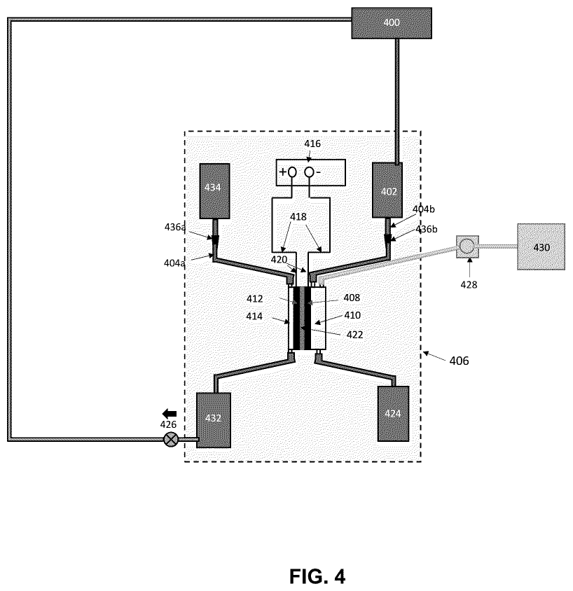

FIG. 4 shows a schematic diagram of coupling a ACPEM/ACCEM and abiotic anode with carbon capture. An electrochemical cell (406), comprising an anode chamber (410) comprising an anode (408), an anode outflow container (424) and a cathode chamber (414) comprising a cathode (412) is connected to a power supply/potentiostat (416) that is electrically connected to the anode (408) and cathode (412) through electrical wires (418) to apply a potential difference between the anode and the cathode. The cathode (412) comprises an embedded current collector (420) that increases power generation of fuel cells by decreasing cathodic charge transfer impedance. A proton/cation exchange membrane (422) separates the anode chamber (410) and the cathode chamber (414) that allows alkali metal ions formed at the anode (408) to diffuse through the proton/cation exchange membrane (422) towards the cathode (412) to react with hydroxide ions to form alkali metal base. A water container (434) supplies a small stream of water through a first flow controller (436a) and first silicone tube (404a) to the inlet of the cathode chamber (414) to carry resulting alkali metal base to a NaOH solution container (432) that is transferred by a pump (426) to the carbon capture unit (400). Carbon dioxide is captured in a carbon capture unit (400) by reacting with the NaOH to form carbonates that are then directed towards a Na₂CO₃ solution container (402). Carbonates in the Na₂CO₃ solution container are transferred through a second flow controller (436b) and second silicone tube (404b) to the anode chamber (410) of the electrochemical cell (406) to react with the H⁺ ions to release the carbon dioxide. A CO₂ sensor (428) detects carbon dioxide released at the anode (408) that is subsequently transferred to an anode gas storage container (430). Additional flow controllers may be incorporated herein to control liquid phase flow as would be understood by a person skilled in the art.

### Example 1b—ACPEM or ACCEM with a Biotic Anode

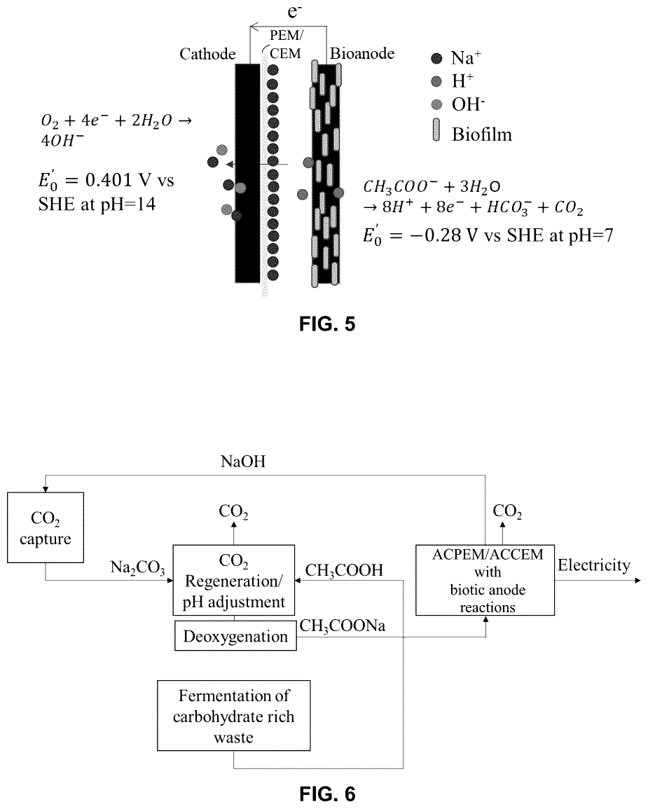

ACPEM/ACCEM with a biotic anode utilizes acetate oxidation reaction catalyzed by anode respiring microorganisms (e.g., Geobacter spp. and Shewanella spp.). These microbes use the electrode as the electron accepter of their acetate oxidation reaction to complete the electrochemical reaction (FIG. 5). As shown in FIG. 5, the resulting cell voltage (cathode—anode potential) is 0.681 V. Acetate oxidation reaction at anode side significantly changes the thermodynamics of the cell when compared with abiotic anode and allows the electrochemical reactions to generate power instead of consuming external power. The tested system showed approximately 2.25 w/m² power output. Without wishing to be bound to theory, such a positive value indicates that instead of consuming energy, power is produced in this system. This system may present operational boundaries. For example, the pH of the anode is to be close to neutral or slightly acidic in the range of pH=6-7 to allow optimum performance of the anode respiring biofilms (Rabaey et al., 2010) as well as releasing of the generated CO₂ (FIG. 2, D)). The concentration of the acetate as well as Na⁺ will be moderate to allow proper functioning of the biofilms (Harrington et al., 2015). Test results showed that the Geobacter sulfurreducens strain KN400 performed well with up to 400 mM of Na⁺ and 200 mM of acetate. The ACPEM/ACCEM is typically outputting a 5-10 A/m² current (Li et al., 2020). These boundaries entail a delicate design when coupling with a carbon capture process (FIG. 6).

FIG. 6 shows the overall scheme of coupling ACPEM/ACCEM and biotic anode with carbon capture.

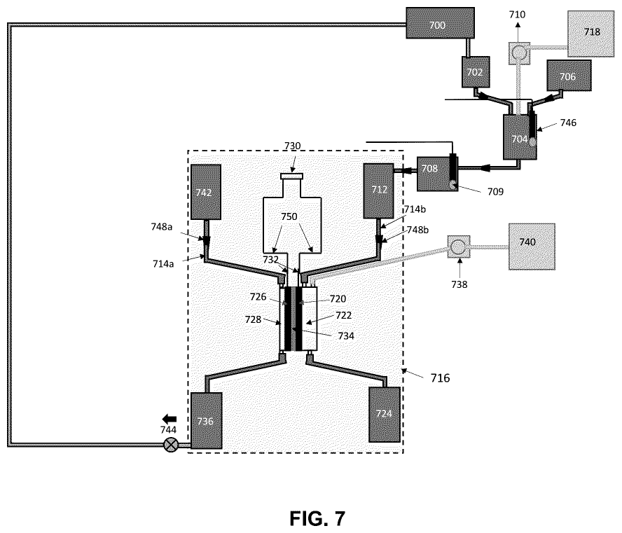

FIG. 7 shows a schematic diagram of coupling an ACPEM/ACCEM with a biotic anode with carbon capture. An electrochemical cell (716), comprising an anode chamber (722) comprising an anode (720), an anode outflow container (724) and a cathode chamber (728) comprising a cathode (726), is connected to an electrical load or battery (730) that is electrically connected to the anode (720) and the cathode (726) through electrical wires (750) to apply a potential difference between the anode and the cathode. The cathode (726) comprises an embedded current collector (732) that increases power generation of fuel cells by decreasing cathodic charge transfer impedance. A proton/cation exchange membrane (734) separates the anode chamber (722) and the cathode chamber (728) that allows alkali metal ions formed at the anode to diffuse through the proton/cation exchange membrane (734) towards the cathode to react with hydroxide ions to form alkali metal base. A water container (742) supplies a small stream of water through a first flow controller (748a) and first silicone tube (714a) to the inlet of the cathode chamber (728) to carry resulting alkali metal base to a NaOH solution container (736) that is transferred by a pump (744) to the carbon capture unit (700). Carbon dioxide is captured in a carbon capture unit (700) by reacting with the NaOH to form carbonates that are then directed towards a Na₂CO₃ solution container (702). The carbonates are directed to a first waste sodium acetate solution (704) to react with waste organic acid solution (706) to produce purified carbon dioxide that is detected by a CO₂ sensor (710). The pH of the waste sodium acetate solution (704) is monitored with a pH sensor (746). Generated CO₂ is then transferred to a CO₂ storage container (718). The first waste sodium acetate solution (704) is deoxygenated with a deoxygenation unit (708) having a dissolved oxygen sensor (709) upon which deoxygenated waste sodium acetate solution (712) is transferred through a second flow controller (748b) and a second silicone tube (714b) back to the anode chamber (722) of the electrochemical cell (716) to react with the H⁺ ions to release the carbon dioxide. A CO₂ sensor (738) detects carbon dioxide released at the anode (720) that is subsequently transferred to CO₂ gas storage container (740). Additional flow controllers may be incorporated herein to control liquid phase flow, for example between the waste organic solution (706) and the first waste sodium acetate solution (704) as would be understood by a person skilled in the art.

A carbohydrate rich organic waste source such as those from sugarcane processing, potato processing, fruit processing and/or the paper mill industry is subjected to fermentation to produce volatile fatty acids (VFAs, e.g., acetic acid) solutions. Other sources of VFAs and their precursors include Fisher-Tropsch reaction water, pyroligneous acid, agricultural waste and waste from sustainable forestry management. Such solutions acidified Na₂CO₃ solution from the CO₂ capture process and allowed the release of CO₂. The primary content in the resulting solution was acetate, after dilution to a suitable concentration (˜200 mM) that serves as an excellent fuel (carbon and energy source) for the anode respiring microbes (e.g., Geobacter spp.). Following the mechanisms illustrated in FIG. 6, the ACPEM/ACCEM with biotic anode system generates voltage/current while allowing the regeneration of NaOH solution. The generated H⁺in the anode is of lower concentration compared to the conditions in ACPEM/ACCEM with abiotic anode, which is buffered against both the CH₃COO⁻/CH₃COOH and HCO₃⁻/H₂CO₃ buffer systems, maintaining a pH around 6.5. At slightly acidic conditions, a larger portion of the produced CO₂ from the acetate oxidation reaction can be released and harvested through the off-gas collection, when compared with the neutral or alkaline conditions.

In another embodiment, an electrochemical cell, comprising an anode chamber comprising an anode, an anode outflow container and a cathode chamber comprising a cathode, is connected to an electrical load or battery that is electrically connected to the anode and the cathode through electrical wires to apply a potential difference between the anode and the cathode. The cathode comprises an embedded current collector that increases power generation of fuel cells by decreasing cathodic charge transfer impedance. A proton/cation exchange membrane separates the anode chamber and the cathode chamber that allows alkali metal ions formed at the anode to diffuse through the proton/cation exchange membrane towards the cathode to react with hydroxide ions to form alkali metal base. A water container supplies a small stream of water through a first flow controller and first silicone tube to the inlet of the cathode chamber to carry resulting alkali metal base to a NaOH solution container that is transferred by a pump to the carbon capture unit. Carbon dioxide is captured in a carbon capture unit by reacting with the NaOH to form carbonates that are then directed towards a Na₂CO₃/NaHCO₃ solution container. The carbonates are directed to a neutralization reactor to react with an organic acid solution, particularly acetic acid, to produce the first stream of CO₂ that is detected by a CO₂ sensor. The pH of the waste sodium acetate solution is monitored with a pH sensor. The first stream of CO₂, which is from air capture, is then transferred to a CO₂ storage container. The neutralized sodium acetate solution is deoxygenated with a deoxygenation unit when necessary. A dissolved oxygen sensor is equipped with the deoxygenation unit. The (deoxygenated) sodium acetate solution is transferred through a second flow controller and a second silicone tube back to the anode chamber of the electrochemical cell to be oxidized by anode respiring microorganisms and to react with the H⁺ ions to release the second stream of CO₂. The second stream of CO₂ is biogenic and from acetate oxidation. A CO₂ sensor detects carbon dioxide released at the anode that is subsequently transferred to CO₂ gas storage container. Additional flow controllers may be incorporated herein to control liquid phase flow, for example organic acid solution and the neutralized sodium acetate solution as would be understood by a person skilled in the art.

The hydrogen ions that are used to release the first stream of CO₂ can be from organic acid (e.g., acetic acid) sourced from carbohydrate rich waste streams from pulp and paper processing, Fischer-Tropsch reaction, biomass pyrolysis, sugar processing, potato processing etc. This leads to waste reduction from these industries, while providing high purity CO₂ capture from both air capture and acetate oxidation. The captured CO₂ can be sequestrated for emission reduction credits or utilized for beverage making, concrete curing, dry ice production, and aviation fuel synthesis, etc. Overall, this approach allows valorization of waste streams into values for the industries. It can also be sourced from ethanol production. After conversion a fraction of ethanol to acetic acid to drive emission reduction from CO₂ removed from both air capture and acetate oxidation, the carbon intensity scores of the remaining ethanol-based fuel products can be significantly reduced to boost their values.

Following the neutralization, the primary component in the resulting solution was acetate, after dilution to a suitable concentration (˜200 mM) that serves as an excellent fuel (carbon and energy source) for the anode respiring microbes (e.g., Geobacter spp.). Following the mechanisms illustrated in FIG. 6, the ACPEM/ACCEM with biotic anode system generates voltage/current while allowing the regeneration of NaOH solution.

Acetate itself is oxidized into CO₂ anaerobically at the anode by the anode aspiring microorganisms, which allows a second CO₂ stream to be evolved and captured. This increases the total quantity of CO₂ captured per cycle. The generated H⁺in the anode is of lower concentration compared to the conditions in ACPEM/ACCEM with abiotic anode, which is buffered against both the CH₃COO⁻/CH₃COOH and HCO₃⁻/H₂CO₃ buffer systems, maintaining a pH around 6.5. At slightly acidic conditions, a larger portion of the produced CO₂ from the acetate oxidation reaction can be released and harvested through the off-gas collection. Additionally, rough vacuum can be applied to extract more CO₂ that is dissolved in the solution by leveraging Henry's law. In the testing conditions, a power output of 2.25 w/m² was observed, which drove the generation of 247 L of 1M NaOH solution per kwh. The captured CO₂ from both streams were found with purity >97%. Steady performance was observed for 3 months of operation.

### Example 2—CO₂ Capture Coupled with Bioelectrochemical Methane Production (BEMP)

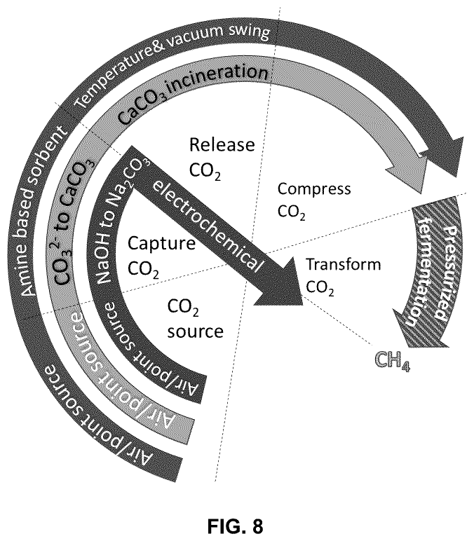

The BEMP as well as biological methane synthesis have been both traditionally operated with gaseous CO₂. However, as illustrated in FIG. 8, relying on the generation of gaseous CO₂ adds to the energy consumption and the cost of the operation. In this design, the CO₂ from air or a point source can be converted to Na₂CO₃ which can be directly subjected to BEMP after pH adjustment and deoxygenation. Following BEMP, the resulting Na⁺ rich solution can then be recovered as NaOH through the ACPEM/ACCEM with a biotic anode process as illustrated in FIG. 6.

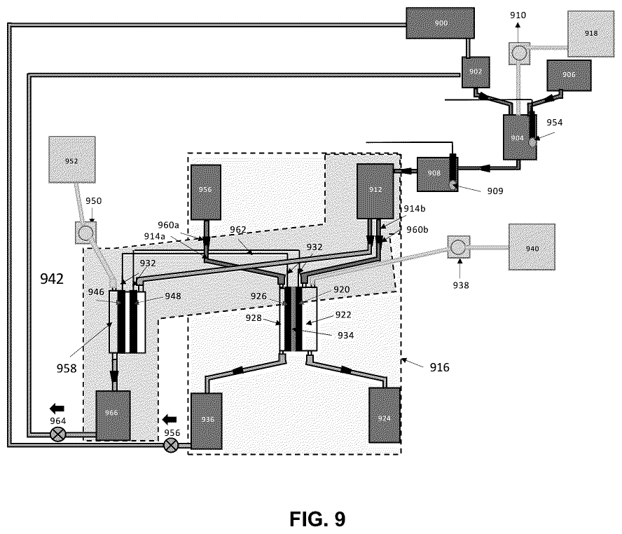

FIG. 9 shows a schematic diagram of coupling a ACPEM/ACCEM and biotic anode to a BEMP module. An electrochemical cell (916), comprising an anode chamber (922) comprising an anode (920), an anode outflow container (924) and a cathode chamber (928) comprising a cathode (926) is connected to a power supply such as a load/battery (not shown) or BEMP. The cathode (926) comprises an embedded current collector (932) that increases power generation of fuel cells by decreasing cathodic charge transfer impedance. A proton/cation exchange membrane (934) separates the anode chamber (922) and the cathode chamber (928) that allows alkali metal ions formed at the anode to diffuse through the proton/cation exchange membrane (934) towards the cathode to react with hydroxide ions to form alkali metal base. A water container (956) supplies a small stream of water through a first flow controller (960a) and a first silicone tube (914a) to the inlet of the cathode chamber (928) to carry resulting alkali metal base to a NaOH solution container (936) that is transferred by a pump (956) to the carbon capture unit (900). Carbon dioxide is captured in a carbon capture unit (900) by reacting with the NaOH to form carbonates that are then directed towards a Na₂CO₃ solution container (902). The carbonates are directed to a first waste sodium acetate solution (904) to react with waste organic acid solution (906) to produce purified carbon dioxide that is detected by a CO₂ sensor (910). The pH of the waste sodium acetate solution (904) is monitored with a pH sensor (954). Generated CO₂ is then transferred to a CO₂ storage container (918). The waste sodium acetate solution (904) is deoxygenated with a deoxygenation unit (908) having a dissolved oxygen sensor (909) upon which deoxygenated waste sodium acetate solution (912) is transferred through a second flow controller (960b) and a second silicone tube (914b) to the anode chamber (922) of the electrochemical cell (916) to react with the H⁺ ions to release the carbon dioxide. A CO₂ sensor (938) detects carbon dioxide released at the anode (920) that is subsequently transferred to CO₂ gas storage container (940). Deoxygenated carbonate solution of (912) may also be transferred directly to a BEMP (942) powered by the ACPEM/ACCEM biotic module. The BEMP module comprises an electrochemical cell (958) having an anode (946) and a cathode (948) wherein acetates are reduced to CH₄ and an anode outflow container (966) collects anode carbonate containing effluent that is subsequently pumped back to the Na₂CO₃ solution container (902). Generated CH₄ is detected by a CH₄ sensor (950) and stored in a CH₄ storage container (952). Additional flow controllers may be incorporated herein to control liquid phase flow, for example between the waste organic solution (906) and the waste sodium acetate solution (904) as would be understood by a person skilled in the art.

Without being bound to theory, this operational route also has the advantages of 1) bypassing the kinetic limiting step of the mass transfer of gaseous CO₂ to the liquid phase (Simon et al., 2018); 2) higher effective CO₂ concentration (buffered in bicarbonate form) leading to reduced Gibbs free energy for CO₂ reduction; and 3) bicarbonate serving as cost-effective buffer and improving conductivity for a bioelectrochemical system (Fan et al., 2007; Wang et al., 2022).

The involvement of Na₂CO₃/NaHCO₃ demands the biocatalysts (methanogens) to be moderately Na⁺ tolerant. Table 1 summarizes the methanogens capable of adapting to a Na₂CO₃/NaHCO₃ based BEMP. Among these species, Methanococcus maripaludis (mesophilic), Methanobthermobacter marburgenesis, Methanothermococcus okinawensis (thermophilic), and Methanotorris igneus (hyperthermophilic) are the most potent producers generating methane at the highest rates. These strains can be spiked to the mixed culture to allow effective methane generation. The concentration of Na₂CO₃/NaHCO₃ added to the system varies according to the physiology of the biocatalysts, while at least a level of 200 mM is recommended.

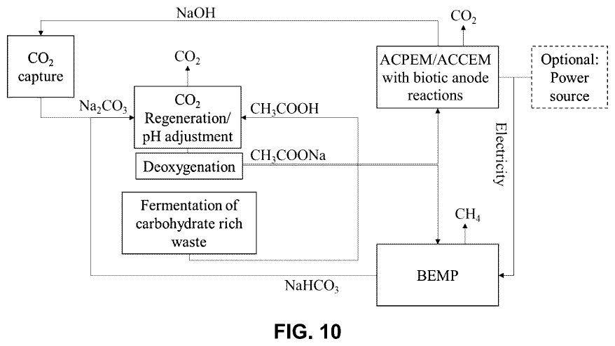

The Na₂CO₃/NaHCO₃ based BEMP is suitable to work in conjunction with ACPEM/ACCEM and biotic anode-based CO₂ capture (FIG. 10). Without wishing to be bound to theory, the primary advantage of coupling these two modules together lies in that the electricity generated from ACPEM/ACCEM with a biotic anode can be utilized at BEMP to reduce bicarbonate to CH₄, resulting in a close to zero energy input system for simultaneous CO₂ capture and CH₄ production with the added benefit of waste reduction. This is a significant advantage over standalone BEMP systems that draw electricity from the grid (e.g., (McGinnis & Herrmann-nowosielski, 2022), and run on higher operational cost. The acetate produced following CO₂ regeneration can be used to supply electrons and protons/cations at the anode (Equations 2-3):

$$Cathode : {HCO}_{3}^{-} + 9 {H}^{+} + 8 {e}^{-} \to {CH}_{4} + 3 {H}_{2} O \qquad ( 2 )$$

$$E ( V ) vs SHE = - 0.24 V at pH = 7$$

$$Anode : {CH}_{3} {COO}^{-} + 3 {H}_{2} O \to 8 {H}^{+} + 8 {e}^{-} + {HCO}_{3}^{-} + {CO}_{2}$$

$$E ( V ) vs SHE = - 0.28 V at pH = 7 \qquad ( 3 )$$

Without wishing to be bound to theory, although the theoretical cell voltage of this pair of reactions is −0.24V−(−0.28V)=0.04V (>0), the reaction is not spontaneous, due to the overpotentials in the system. Therefore, an external power source such as ACPEM/ACCEM and biotic anode, or power from the grid can be used to drive the reaction. Acetoclastic methanogenesis and hydrogen evolution reactions also occurred in this BEMP. The equilibriums of these reactions reached at various applied voltage/current levels and PH levels. A slightly alkaline condition (pH=7.5-8) is maintained to allow most of the generated CO₂ to stay in the bicarbonate form and release a high purity CH₄ (>90%). The resulting bicarbonate solution can be recycled through the CO₂ regeneration/pH adjustment process (FIG. 10). When compared with an anaerobic digestor, this process has better pH management by taking advantage of bicarbonate generated from CO₂ capture. It can also separate CO₂ and CH₄ gaseous during the process allowing the generation of two valuable products instead of a mixture as in biogas.

TABLE 1
Methanogen biocatalyst candidates selected for BEMP coupled with CO₂
capture (Lyu et al., 2018; Mauerhofer et al., 2021). Darker shades highlight the most
potent mesophilic, thermophilic, and hyperthermophilic methanogens.

| | | | | | | | |
|---|---|---|---|---|---|---|---|
| | Methano- | Required | | | | | CH₄ evolution |
| | genesis | organic | | pH | NaCl | Doubting | rate |
| Species | substrates | compounds | T ºC. | (Optimum) | range | time (h) | (mmol/L/h) |

Methanobacterium

| | | | | | | | |
|---|---|---|---|---|---|---|---|
| aahusense | H₂ + CO₂ | None | 5-48 | 5-9 | 0.6-5.4 | nd | 1.05E−02 |
| | | | (45) | (7.5-8) | | | |
| alcaliphilum | H₂ + CO₂ | TP or YE | 25-45 | 7-9.9 | nd | nd | nd |
| | | | (37) | (8.1-9.1) | | | |
| beijingense | H₂ + CO₂, | YE | 25-50 | 6.5-8 | 0-3 | 14 | 1.62E−02 |
| | formate | | (37) | (7.2) | | | |
| ferruginis | H₂ + CO₂, | None | 20-45 | 5.5-9.0 | 0-7 | 18.5 | 1.66E−02 |
| | 2-propanol, | | (40) | (6.0-7.5) | | | |
| | 2-butanol, | | | | | | |
| | cyclo- | | | | | | |
| | pentanol | | | | | | |
| kanagiense | H₂ + CO₂ | None | 15-45 | 6.5-9.6 | 0-7 | 21 | 2.34E−02 |
| | | | (40) | (7.5-8.5) | | | |
| lacus | H₂ + CO₂, | None | 14-41 | 5-8.5 | 0-2.3 | 22 | 6.39E−03 |
| | methanol | | (30) | (6.5) | | | |
| movilense | H₂ + CO₂, | None | 0-44 | 6.2-9.9 | 0.1 | 79.2 | 1.84E−02 |
| | formate, | | (33) | (7.4) | 3.5 | | |
| | 2-propanol, | | | | | | |
| | 2-butanol | | | | | | |

Methanobrevibacter

| | | | | | | | |
|---|---|---|---|---|---|---|---|
| boviskoreani | H₂ + CO₂, | YE, COM, | 35-45 | 5.5-8.0 | 0.6-3.0 | nd | 1.78E−02 |
| | formate | FA | (37-40) | (6.5-7.0) | | | |
| millerae | H₂ + CO₂, | Ac, YE or | 33-43 | 5.5-10.0 | up to | nd | 1.33E−02 |
| | formate | TP | (36-42) | (7.0-8.0) | 2.6 | | |

Methanobthermobacter

| | | | | | | | |
|---|---|---|---|---|---|---|---|
| crinale | H₂ + CO₂ | ac | 45-80 | 6.9-8.0 | 0-4 | nd | 8.20E−03 |
| | | | (65) | (6.9) | | | |
| 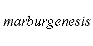 | 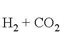 | 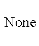 | 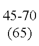 | 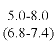 | 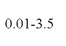 | 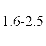 | 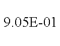 |
| | | | | 6.8 7.4 | | | |
| thermoauto- | H₂ + CO₂, | None | 40-75 | 6.0-8.8 | 0.01-3.5 | 3 | 1.43E−02 |
| trophicus | formate | | (65-70) | (7.2-7.6) | | | |
| thermoflexus | H₂ + CO₂, | CoM | 47-70 | 7.5-8.5 | 0.1-3 | 3.5 | 1.70E−02 |
| | formate | | (55) | (7.9-8.2) | | | |

Methanocaldococcus

| | | | | | | | |
|---|---|---|---|---|---|---|---|
| fervens | H₂ + CO₂ | None | 48-92 | 5.5-7.6 | 0.5-5 | 0.3-0.5 | nd |
| | | | (85) | (6.5) | | | |
| bathoardescens | H₂ + CO₂ | None | 58-90 | 4.5-9.0 | 1.6-7.4 | 0.3 | nd |
| | | | (82) | (7.0) | | | |

Methanotorris

| | | | | | | | |
|---|---|---|---|---|---|---|---|
| 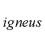 | 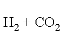 | 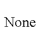 | 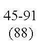 | 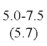 | 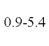 | 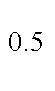 | 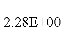 |
| formicicus | H₂ + CO₂, | None | 55-83 | 6.0-8.5 | 0.4-6.0 | 0.5 | nd |
| | formate | | (75) | (6.7) | | | |

Methanococcus

| | | | | | | | |
|---|---|---|---|---|---|---|---|
| vannielii | H₂ + CO₂, | None | 20-45 | 6.5-8.0 | 0.3-5 | 8 | 2.09E−02 |
| | formate | | (35-40) | (7-8) | | | |
| voltae | H₂ + CO₂, | None | 20-45 | 6.5-8.0 | 0.6-6 | 3 | 2.02E−02 |
| | formate | | (35-40) | (6.5-7.0) | | | |
| 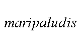 | 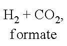 | 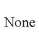 | 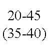 | 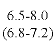 | 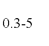 | 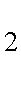 | 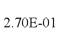 |
| aeolicus | H₂ + CO₂, | None | 20-45 | 5.5-7.5 | 0.3-6 | 1.3 | 9.18E−03 |
| | formate | | (46) | (7.0) | | | |

Methanothermococcus

| | | | | | | | |
|---|---|---|---|---|---|---|---|
| thermolitho- | H₂ + CO₂, | None | 17-70 | 4.5-8.5 | 0.6-9.4 | 1 | 3.32E−02 |
| trophicus | formate | | (60-65) | (6-7) | | | |
| 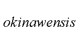 | 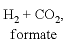 | 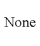 | 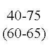 | 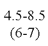 | 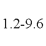 | 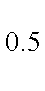 | 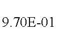 |

Methanofollis

| | | | | | | | |
|---|---|---|---|---|---|---|---|
| formosanus | H₂ + CO₂, | YE, TP | 20-42 | 5.6-7.3 | 3 | 36 | nd |
| | formate | | (40) | (6.6-7.0) | | | |
| liminatans | H₂ + CO₂, | ac | 15-44 | nd (7) | 0-3.5 | 7.5 | nd |
| | formate, | | (40) | | | | |
| | 2-propanol, | | | | | | |
| | 2-butanol, | | | | | | |
| | cyclo- | | | | | | |
| | pentanol | | | | | | |

Methanogenium

| | | | | | | | |
|---|---|---|---|---|---|---|---|
| frigidum | H₂ + CO₂, | ac | −12-18 | 6.5-7.9 | 2-3.5 | 69.6 | 1.73E−02 |
| | formate | | (15) | (7.5-7.9) | | | |
| marinum | H₂ + CO₂, | ac | 5-25 | 5.5-7.5 | 1.5-7.3 | 42 | 1.23E−02 |
| | formate | | (25) | | | | |

Methanolaciania

| | | | | | | | |
|---|---|---|---|---|---|---|---|
| petrolearia | H₂ + CO₂, | ac | 28-43 | 5.3-8.2 | 1-3 | 10 | 2.20E−02 |
| | formate, | | (35-40) | (7.0) | | | |
| | 2-propanol | | | | | | |

For the carbon capture solution to be an effective feedstock for BEMP, significant reduction of dissolved oxygen level in the CO₂ capture solution is crucial (FIGS. 6 and 10), as exposing to high level of oxygen is lethal to these biocatalysts. The below section will discuss a biological process that was used to remove dissolve oxygen in the Na₂CO₃/NaHCO₃ solution effectively.

### Example 2a—BEMP with Cost-Effective Deoxygenation

As both anode respiring microbes and methanogens are sensitive to oxygen toxicity, dissolved oxygen in the carbon capture solution should be significantly reduced. A deoxygenation unit was placed after the mixing of VFAs with Na₂CO₃ as shown in FIGS. 6 and 10. To take advantage of the presence of VFAs and now a balanced pH, a biological deoxygenation process was introduced. An aerobic bacterium, Halomonas alkaliphila, with high tolerance to Na₂CO₃/NaHCO₃, cultured to high cell density, was confined in dialysis tubes with 1000 Da cutoff. The cell tubes were used to deoxygenate the mixture of the carbon capture solution and VFA solution generated from the carbohydrate rich organic waste. The VFA solution already came with low dissolved oxygen (DO) through the fermentation process. The carbon capture solution, however, had saturated DO. Since Halomonas alkaliphile grows in the pH of 8-9, the mixture for deoxygenation had lower portion of VFA solution to allow a relatively high pH to support strain respiration. The deoxygenated final product was then combined with more VFA solution to achieve ideal pH values for ACPEM/ACCEM with biotic anode and/or coupling to the BEMP module. These 10 cm L×2 cm DI cell tubes were capable of reducing the DO from saturation with 10% volume (6L total) of air headspace to less than 0.1 mg/L within 10 hrs, while control conditions without cell tubes took 30 hrs to reduce the DO to 0.5 mg/L. A 2-level factorial design was used to examine the influences of mixing speed, number of cell tubs and pH values on oxygen consumption rates and final DO in mg/L (Table 2).

TABLE 2
2-level factorial design to evaluate the influences of mixing
speed, number of cell tubes and pH values on the oxygen consumption
rate and the final dissolved oxygen concentration.

| Study Type | Factorial | Runs | 11 |
|---|---|---|---|
| Design Type | 2 Level | Blocks | No Blocks |
| | Factorial | | |
| Center Points | 3 | | |
| Design Model | 3FI | | |

| Factor | Name | Units | Type | Minimum | Maximum | Mean |
|---|---|---|---|---|---|---|
| A | Mixing speed | RMP | Numeric | 0 | 100 | 50 |
| B | Number of | 1 | Numeric | 1 | 3 | 2 |
| | cell tubes | | | | | |
| C | pH | M | Numeric | 8 | 10 | 9 |

| Response | Name | Units | Response | Name | Units |
|---|---|---|---|---|---|
| Y1 | Oxygen | mg/hr | Y2 | Final DO | mg/L |
| | consumption | | | | |
| | rate | | | | |

The mixing speed was not found as a significant factor on the oxygen consumption and final DO. Static operation showed satisfactory performance while saving energy cost. pH values of 8 and 9 showed more promising outcomes than pH 10. Higher tube numbers lead to improved operational outcomes.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

### REFERENCES

Bouwman, L., Pant, D., Bulut, M., Vanbroekhoven, K. 2018. Alkali-mediated carbon dioxide capture and utilization, (Ed.) E.P. Office.
Fan, Y., Hu, H., Liu, H. 2007. Sustainable power generation in microbial fuel cells using bicarbonate buffer and proton transfer mechanisms. Environmental science & technology, 41 (23), 8154-8158.
Geppert, F., Liu, D., van Eerten-Jansen, M., Weidner, E., Buisman, C., Ter Heijne, A. 2016. Bioelectrochemical power-to-gas: state of the art and future perspectives. Trends in biotechnology, 34 (11), 879-894.
Gong, Y., Wang, Z., Wang, S. 2006. Experiments and simulation of CO₂ removal by mixed amines in a hollow fiber membrane module. Chemical Engineering and Processing: Process Intensification, 45 (8), 652-660.
Harrington, T. D., Babauta, J. T., Davenport, E. K., Renslow, R. S., Beyenal, H. 2015. Excess surface area in bioelectrochemical systems causes ion transport limitations. Biotechnology and bioengineering, 112 (5), 858-866.
Jiang, C., Wang, Y., Xu, T. 2013. An excellent method to produce morpholine by bipolar membrane electrodialysis. Separation and Purification Technology, 115, 100-106.
Keith, D. W., Holmes, G., Angelo, D. S., Heidel, K. 2018. A process for capturing CO₂ from the atmosphere. Joule, 2 (8), 1573-1594.
Li, T., Zhou, Q., Zhou, L., Yan, Y., Liao, C., Wan, L., An, J., Li, N., Wang, X. 2020. Acetate limitation selects Geobacter from mixed inoculum and reduces polysaccharide in electroactive biofilm. Water Research, 177, 115776.
Lyu, Z., Liu, Y., Stams, A., Sousa, D. 2018. Diversity and taxonomy of methanogens. Biogenesis of hydrocarbons, 1-59.
Mauerhofer, L.-M., Zwirtmayr, S., Pappenreiter, P., Bernacchi, S., Seifert, A. H., Reischl, B., Schmider, T., Taubner, R.-S., Paulik, C., Rittmann, S. K.-M. 2021. Hyperthermophilic methanogenic archaea act as high-pressure CH4 cell factories. Communications biology, 4 (1), 1-12.
McGinnis, R., Herrmann-nowosielski, M. 2022. Integrated direct air capture of CO₂ for aqueous electrochemical reduction of CO₂, Google Patents.
Pan, S., Li, H., Liu, D., Huang, R., Pan, X., Ren, D., Li, J., Shakouri, M., Zhang, Q., Wang, M. 2022. Efficient and stable noble-metal-free catalyst for acidic water oxidation. Nature Communications, 13 (1), 1-10.
Penders-van Elk, N. J., Hamborg, E. S., Huttenhuis, P. J., Fradette, S., Carley, J. A., Versteeg, G. F. 2013. Kinetics of absorption of carbon dioxide in aqueous amine and carbonate solutions with carbonic anhydrase. International Journal of Greenhouse Gas Control, 12, 259-268.
Peng, Y., Zhao, B., Li, L. 2012. Advance in post-combustion CO₂ capture with alkaline solution: a brief review. Energy Procedia, 14, 1515-1522.
Rabaey, K., Butzer, S., Brown, S., Keller, J.r., Rozendal, R. A. 2010. High current generation coupled to caustic production using a lamellar bioelectrochemical system. Environmental Science & Technology, 44 (11), 4315-4321.
Sabatino, F., Mehta, M., Grimm, A., Gazzani, M., Gallucci, F., Kramer, G. J., van Sint Annaland, M. 2020. Evaluation of a direct air capture process combining wet scrubbing and bipolar membrane electrodialysis. Industrial & Engineering Chemistry Research, 59 (15), 7007-7020.
Simon, K.-M. R., Seifert, A. H., Bernacchi, S. 2018. Kinetics, multivariate statistical modelling, and physiology of CO₂-based biological methane production. Applied Energy, 216, 751-760.
Sundberg, J., Cameron, L. J., Southon, P. D., Kepert, C. J., Mckenzie, C. J. 2014. Oxygen chemisorption/desorption in a reversible single-crystal-to-single-crystal transformation. Chemical Science, 5 (10), 4017-4025.
Tiwari, B., Noori, M. T., Ghangrekar, M. 2016. A novel low cost polyvinyl alcohol-Nafion-borosilicate membrane separator for microbial fuel cell. Materials chemistry and physics, 182, 86-93.
Valluri, S., Kawatra, S. 2021. Reduced reagent regeneration energy for CO₂ capture with bipolar membrane electrodialysis. Fuel Processing Technology, 213, 106691.
Wang, B., Liu, W., Zhang, Y., Wang, A. 2022. Natural solar intermittent-powered electromethanogenesis towards green carbon reduction. Chemical Engineering Journal, 432, 134369.
Welch, A. J., Dunn, E., DuChene, J. S., Atwater, H. A. 2020. Bicarbonate or carbonate processes for coupling carbon dioxide capture and electrochemical conversion. ACS Energy Letters, 5 (3), 940-945.
Ye, W., Huang, J., Lin, J., Zhang, X., Shen, J., Luis, P., Van der Bruggen, B. 2015. Environmental evaluation of bipolar membrane electrodialysis for NaOH production from wastewater: Conditioning NaOH as a CO₂ absorbent. Separation and purification technology, 144, 206-214.
Zhang, C., Liang, P., Jiang, Y., Huang, X. 2015. Enhanced power generation of microbial fuel cell using manganese dioxide-coated anode in flow-through mode. Journal of Power Sources, 273, 580-583.
Zhou, H., Zhang, H., Zhao, P., Yi, B. 2006. A comparative study of carbon felt and activated carbon based electrodes for sodium polysulfide/bromine redox flow battery. Electrochimica Acta, 51 (28), 6304-6312.

1. An electrochemical system for capturing carbon dioxide from a gas stream, the system comprising:
an electrochemical cell comprising an anode chamber and a cathode chamber, where the cell contains an electrolyte solution;
a cathode exposed to the electrolyte solution in the cathode chamber;
an anode exposed to the electrolyte solution in the anode chamber;
wherein the electrochemical cell is connectable to a power supply for electrically connecting the anode and the cathode to apply a potential difference between the anode and the cathode;
a proton or cation exchange membrane separating the anode chamber and the cathode chamber allowing alkali metal ions formed at the anode to diffuse through the proton or cation exchange membrane towards the cathode to react with hydroxide ions to form an alkali metal base,
a collection chamber to collect the alkali metal base from the cathode chamber;
a source of the gas stream comprising carbon dioxide for contacting with the alkali metal base such that the carbon dioxide reacts with the alkali metal base to form a carbonate, and thus capture the carbon dioxide from the gas stream.

2. The system of claim 1, wherein the cathode is an air cathode or a hydrogen evolution cathode, and wherein the anode is an abiotic anode or a biotic anode.

3. The system of claim 1, wherein the anode is configured to generate an oxygen evolution reaction or an acetate oxidation reaction.

4. The system of claim 1, wherein the anode is a biotic anode configured to generate an acetate oxidation reaction, wherein the acetate oxidation reaction generates power.

5. The system of claim 4, wherein the acetate oxidation reaction at the biotic anode further releases CO₂.

6. The system of claim 4, wherein the acetate oxidation reaction is catalyzed by respiring microorganisms, selected from Geobacter spp and Shewanella spp.

7. The system of claim 1, wherein the electrolyte solution in the anode chamber has a pH from about 5 to about 7, or about 6 to about 7, or about 7, and wherein the electrolyte solution in the cathode chamber has a pH of about 12 to about 14, or about 13 to about 14, or about 14.

8. The system of claim 1, wherein the cathode comprises a carbon material selected from a porous carbon felt, activated carbon, carbon cloth, or VULCAN carbon support and combinations thereof, and is optionally doped with nitrogen, and wherein a film selected from MnO₂, Pt, Fe or Co and combinations thereof is optionally deposited onto the carbon material.

9. The system of claim 1, wherein the electrolyte solution comprises an aqueous solution of a salt of the formula XₙY wherein X is Na⁺, K⁺, or Li⁺, Y is SO₄²⁻, CO₃²⁻, HCO₃⁻ and n is 1 or 2 or a combination thereof, and the alkali metal ions is selected from Na⁺, K⁺, Li⁺ and combinations thereof.

10. The system of claim 1, wherein the potential difference is from about −0.9V to about 0.75V, or −0.85V to about 0.7V, or −0.8V to about 0.65V, or −0.75 to about 0.6V, or about −0.7V to about 0.55V.

11. The system of claim 1, wherein the proton or cation exchange membrane is selected from pure polymer membranes and composite membranes, or is a semipermeable membrane comprising perfluorinated sulfonic acid ionomers, sulfonated poly (ether ether ketone) or polyvinyl alcohol (PVA)-Nafion-borosilicate.

12. The system of claim 1, wherein the gas stream is selected from air, exhaust gas, and flue gas produced during the combustion of fossil fuels, coal, oil or natural gas.

13. The system of claim 1, further comprising a transferrer of the alkali metal base from the collection chamber to a gas contactor configured to bring the gas in contact with the alkali metal base, and optionally a transferrer of the captured carbon dioxide in the form of carbonate to the anode to react the carbonate with hydrogen ions and release the carbon dioxide, a transferrer of the captured carbon dioxide in the form of carbonate to a deoxygenation system and subsequently to the anode to react the carbonate with hydrogen ions and release the carbon dioxide, and/or a transferrer of the captured carbon dioxide in the form of carbonate to a further processing system, wherein the further processing system comprises a transformation, purification or compression system and combinations thereof.

14. The system of claim 13, wherein the transferrers independently operate by mechanical action or gravity, optionally wherein each of the transferrers is independently selected from a peristatic pump, a pressure driven flow control pump, a diaphragm pump, a centrifugal pump and combinations thereof.

15. The system of claim 13, wherein the transformation system produces compounds selected from a biomethane, syngas, formic acid and combinations thereof.

16. A method for capturing carbon dioxide from a gas stream, comprising:
applying a potential difference between an anode and a cathode in an electrochemical cell containing an electrolytic solution such that alkali metal ions are formed in the electrolytic solution at the anode,
permitting the alkali metal ions to diffuse through a proton or cation exchange membrane between the anode and the cathode, towards the cathode to react with hydroxide ions to form an alkali metal base,
separating the alkali metal base from the electrolytic solution,
contacting the gas stream comprising carbon dioxide with the alkali metal base such that the carbon dioxide reacts with the alkali metal base to form a carbonate, thus capturing the carbon dioxide from the gas stream.

17. The method of claim 16, wherein applying the potential difference generates an oxygen evolution reaction at the anode or an acetate oxidation reaction at the anode.

18. The method of claim 16, wherein contacting the gas stream comprising carbon dioxide with the alkali metal base comprises transferring the alkali metal base to a gas contactor to bring the gas in contact with the alkali metal base, and optionally further comprising transferring the captured carbon dioxide in the form of carbonate to the anode to react the carbonate with hydrogen ions and release the carbon dioxide, further comprising transferring the captured carbon dioxide in the form of carbonate to a deoxygenation system and subsequently to the anode to react the carbonate with hydrogen ions and release the carbon dioxide, and/or further comprising transferring the captured carbon dioxide in the form of carbonate to a further processing system, wherein the further processing system comprises a transformation, purification or compression system and combinations thereof.

19. The method of claim 18, wherein the transformation system produces compounds selected from a biomethane, syngas, formic acid and combinations thereof.

20. An electrochemical system for capturing carbon dioxide from a gas stream and generating methane, the system comprising:
an electrochemical cell comprising an anode chamber and a cathode chamber, where the cell contains an electrolyte solution;
a cathode exposed to the electrolyte solution in the cathode chamber;
an anode exposed to the electrolyte solution in the anode chamber;
a power supply electrically connected to the anode and the cathode configured to apply a potential difference between the anode and the cathode;
a proton or cation exchange membrane separating the anode chamber and the cathode chamber allowing alkali metal ions formed at the anode to diffuse through the proton or cation exchange membrane towards the cathode to react with hydroxide ions to form an alkali metal base,
a collection chamber to collect the alkali metal base from the cathode chamber;
a source of the gas stream comprising carbon dioxide for contacting with the alkali metal base such that the carbon dioxide reacts with the alkali metal base to form a carbonate, and thus capture the carbon dioxide from the gas stream,
a deoxygenation unit to remove dissolved oxygen from the carbonate solution to produce a deoxygenated carbonate solution,
and
a biomethane production module configured to receive the deoxygenated carbonate solution, the biomethane production module comprising biocatalysts to react with the carbonate to produce methane.
